(19) United States
(12) Patent Application Publication
(10) Pub. No.: US 2012/0296398 A1
(43) Pub. Date: Nov. 22, 2012

(54) TELEMETRY WAND

(75) Inventor: Daniel Aghassian, Glendale, CA (US)

(73) Assignee: BOSTON SCIENTIFIC NEUROMODULATION CORPORATION, Valencia, CA (US)

(21) Appl. No.: 13/474,046

(22) Filed: May 17, 2012

Related U.S. Application Data
(60) Provisional application No. 61/488,287, filed on May 20, 2011.

(51) Int. Cl. A61N 1/36

(52) U.S. Cl. 607/60

(57) ABSTRACT

A telemetry wand to facilitate communication between a programmer and a neurostimulator device. The telemetry wand comprises an antenna, telemetry circuitry configured for transmitting signals between the programmer and the neurostimulator device via the antenna coil, a threaded screw receptacle configured for receiving a bolt of a conventional camera tripod, and a housing carrying the antenna coil, the telemetry circuitry, and the threaded screw receptacle.

### RELATED APPLICATION DATA

The present application claims the benefit under 35 U.S.C. §119 to U.S. provisional patent application Ser. No. 61/488,287, filed May 20, 2011. The foregoing application is hereby incorporated by reference into the present application in its entirety.

### FIELD OF THE INVENTION

The present invention relates to telemetry devices, and more particularly, to programmable wands for providing telemetric communication between implantable devices and programming devices.

### BACKGROUND OF THE INVENTION

Implantable neurostimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) systems have long been accepted as a therapeutic modality for the treatment of chronic pain syndromes, and the application of tissue stimulation has begun to expand to additional applications such as angina pectoralis and incontinence. Deep Brain Stimulation (DBS) has also been applied therapeutically for well over a decade for the treatment of refractory chronic pain syndromes, and DBS has also recently been applied in additional areas such as movement disorders and epilepsy. Further, Functional Electrical Stimulation (FES) systems such as the Freehand system by NeuroControl (Cleveland, Ohio) have been applied to restore some functionality to paralyzed extremities in spinal cord injury patients. Furthermore, in recent investigations Peripheral Nerve Stimulation (PNS) systems have demonstrated efficacy in the treatment of chronic pain syndromes and incontinence, and a number of additional applications are currently under investigation. Occipital Nerve Stimulation (ONS), in which leads are implanted in the tissue over the occipital nerves, has shown promise as a treatment for various headaches, including migraine headaches, cluster headaches, and cervicogenic headaches.

These implantable neurostimulation systems typically include one or more electrode carrying neurostimulation leads, which are implanted at the desired stimulation site, and an implantable pulse generator (IPG) implanted remotely from the stimulation site, but coupled either directly to the neurostimulation lead(s) or indirectly to the neurostimulation lead(s) via a lead extension. Thus, electrical pulses can be delivered from the IPG to the neurostimulation leads to stimulate the tissue and provide the desired efficacious therapy to the patient.

The neurostimulation system may further comprise a handheld external control device in the form of a remote control (RC) to remotely instruct the IPG to generate electrical stimulation pulses in accordance with selected stimulation parameters. A typical stimulation parameter set may include the electrodes that are acting as anodes or cathodes, as well as the amplitude, duration, and rate of the stimulation pulses. The RC may, itself, be programmed by a clinician, for example, by using a clinician's programmer (CP), which typically includes a general purpose computer, such as a laptop, with a programming software package installed thereon. Typically, the RC can only control the IPG in a limited manner (e.g., by only selecting a program or adjusting the pulse amplitude or pulse width), whereas the CP can be used to control all of the stimulation parameters, including which electrodes are cathodes or anodes. In any event, once the IPG is programmed, it is capable providing the required neurostimulation therapy to the patient without being actively linked to the RC or CP. The most important use of the CP is in the operating room during a trial or permanent surgery. For this use, the CP and any other device used to communicate with the IPG must be deployed quickly and operate reliably without interfering or extending the time required to complete the surgical procedure.

Typically, due to the short-range telemetric requirements of an IPG and the relatively large size of the CP, a portable communications device must be placed in close proximity to the IPG, e.g., in contact with the patient's skin right above the implanted IPG. For example, the RC, itself, may be used by the CP to telemetrically communicate with the IPG. However, because the RC is not a device dedicated to facilitate communication between the CP and the IPG, it is difficult to use and requires several time consuming steps to set up before it can be used. First, the user must ensure a new set of batteries are inserted into the RC, and then must connect several cables to a wireless port (e.g., an Infrared (IR) port of the RC to a serial port of the CP. Then the user must navigate through several menus to place the RC in a special mode (pass through mode) to enable the CP to communicate with the IPG via the RC. Furthermore, since RC is primarily a patient device, there is no provision to position the RC in the required orientation for communication with the IPG. Typically, the RC is laid on a table next to the patient or held by the user. However, because the antenna of the RC must be in an optimum position and orientation relative to the antenna of the IPG, laying the RC on the table may not achieve this optimum positional relationship between the RC and the IPG. If the RC is held by the user, another person must be present to manipulate the controls on the CP 18. Furthermore, when there are difficulties with the telemetry, the RC does not provide a quick and easy to understand status facilitating troubleshooting of the programming system.

There, thus, remains a need for an improved telemetric device that facilitates communication between a CP and an IPG.

### SUMMARY OF THE INVENTION

In accordance with the present invention, a telemetry wand to facilitate communication between a programmer and a neurostimulator device is provided. The telemetry wand comprises an antenna (e.g., a coil), telemetry circuitry configured for transmitting signals between the programmer and the neurostimulator device via the antenna coil, a threaded screw receptacle configured for receiving a bolt of a conventional camera tripod, and a housing carrying the antenna coil, the telemetry circuitry, and the threaded screw receptacle. In an optional embodiment, the telemetry wand further comprises a port (e.g., a USB port) configured for receiving a connector of a telemetry cable. In another optional embodiment, the telemetry wand comprises at least one indicator (e.g., a power on/off indicator, an uplink indicator, a downlink indicator, and a Received Signal Strength Indication (RSSI) indicator) for indicating a communication status with the neurostimulator. The indicator(s) may be, e.g., a visual indicator or an audio indicator.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is plan view of one embodiment of a spinal cord stimulation (SCS) system arranged in accordance with the present inventions;

FIG. 2 is a plan view of an implantable pulse generator (IPG) and two neurostimulation leads used in the SCS system of FIG. 1;

FIG. 3 is a perspective view of a telemetry wand used in the SCS system of FIG. 1;

FIG. 4 is another perspective view of the telemetry wand of FIG. 3;

FIG. 5 is a front view of the telemetry wand of FIG. 3;

FIG. 6 is a side view of the telemetry wand of FIG. 3;

FIG. 7 is a rear view of the telemetry wand of FIG. 3;

FIG. 8 is a perspective view of the telemetry wand of FIG. 3 without the top housing portion; and

FIG. 9 is a perspective view of the telemetry wand of FIG. 3 used mounted on a conventional camera tripod.

### DETAILED DESCRIPTION OF THE EMBODIMENTS

The description that follows relates to a spinal cord stimulation (SCS) system. However, it is to be understood that while the invention lends itself well to applications in SCS, the invention, in its broadest aspects, may not be so limited. Rather, the invention may be used with any type of implantable electrical circuitry used to stimulate tissue. For example, the present invention may be used as part of neurostimulation system, such as a pacemaker, a defibrillator, a cochlear stimulator, a retinal stimulator, a stimulator configured to produce coordinated limb movement, a cortical stimulator, a deep brain stimulator, peripheral nerve stimulator, microstimulator, or in any other neural stimulator configured to treat urinary incontinence, sleep apnea, shoulder sublaxation, headache, etc.

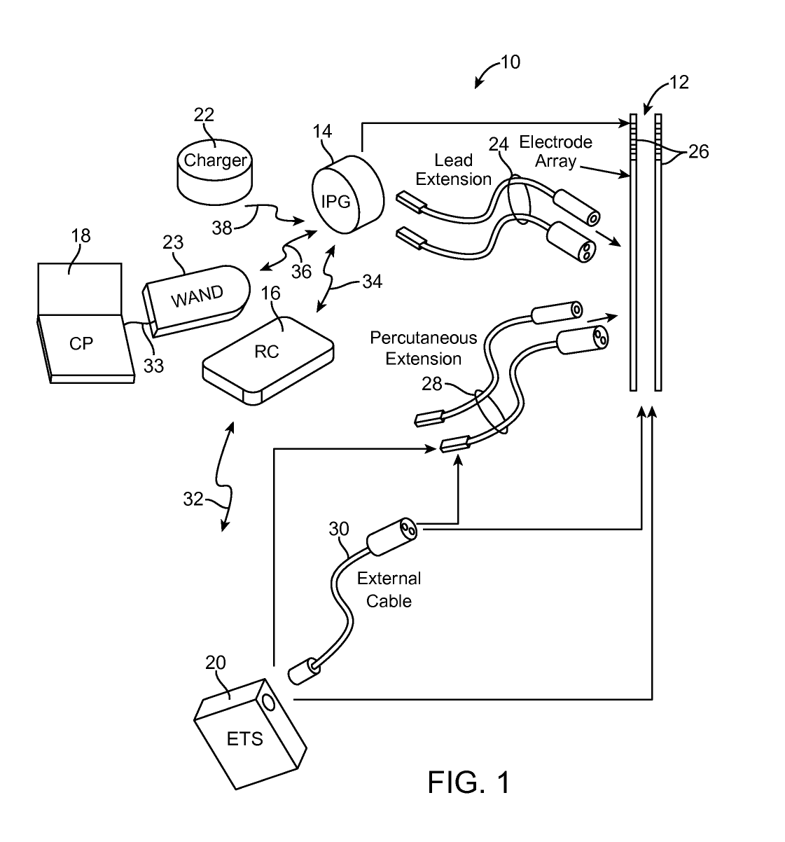

Turning first to FIG. 1, an exemplary SCS system 10 generally comprises a plurality of percutaneous neurostimulation leads 12 (in this case, two leads 12(1) and 12(2)), an implantable pulse generator (IPG) 14, an external remote control (RC) 16, a Clinician's Programmer (CP) 18, an External Trial Stimulator (ETS) 20, an external charger 22, and a telemetry wand 23.

The IPG 14 is physically connected via two lead extensions 24 to the neurostimulation leads 12, which carry a plurality of electrodes 26 arranged in an array. The IPG 14 includes a replenishable power source, telemetry circuitry, and pulse generation circuitry that delivers electrical stimulation energy in the form of a pulsed electrical waveform (i.e., a temporal series of electrical pulses) to the electrode array 26 in accordance with a set of stimulation parameters. The IPG 14 and neurostimulation leads 12 can be provided as an implantable neurostimulation kit, along with, e.g., a hollow needle, a stylet, a tunneling tool, and a tunneling straw. Further details discussing implantable kits are disclosed in U.S. Patent Publication 2009/0216306, entitled “Temporary Neurostimulation Lead Identification Device,” which is expressly incorporated herein by reference.

The ETS 20 may also be physically connected via percutaneous lead extensions 28 or external cable 30 to the neurostimulation lead 12. The ETS 20, which has similar pulse generation circuitry as the IPG 14, also delivers electrical stimulation energy in the form of a pulse electrical waveform to the electrode array 26 in accordance with a set of stimulation parameters. The major difference between the ETS 20 and the IPG 14 is that the ETS 20 is a non-implantable device that is used on a trial basis after the neurostimulation lead 12 has been implanted and prior to implantation of the IPG 14, to test the responsiveness of the stimulation that is to be provided. Further details of an exemplary ETS are described in U.S. Pat. No. 6,895,280, which is expressly incorporated herein by reference.

The RC 16 may be used to telemetrically control the ETS 20 via a bi-directional RF communications link 32. Once the IPG 14 and neurostimulation lead 12 is implanted, the RC 16 may be used to telemetrically control the IPG 14 via a bi-directional RF communications link 34. Such control allows the IPG 14 to be turned on or off and to be programmed with different stimulation programs after implantation. Once the IPG 14 has been programmed, and its power source has been charged or otherwise replenished, the IPG 14 may function as programmed without the RC 16 being present.

The CP 18 provides clinician detailed stimulation parameters for programming the IPG 14 and ETS 20 in the operating room and in follow-up sessions. The CP 18 may perform this function by indirectly communicating with the IPG 14 or ETS 20, through the telemetry wand 23. In particular, the CP 18 communicates with the telemetry wand 23 via a telemetry cable 33, and the telemetry wand 23 communicates with the IPG 14 via a bi-directional RF link 36.

The external charger 22 is a portable device used to transcutaneously charge the IPG 14 via an inductive link 38. Once the IPG 14 has been programmed, and its power source has been charged by the external charger 22 or otherwise replenished, the IPG 14 may function as programmed without the RC 16 or CP 18 being present.

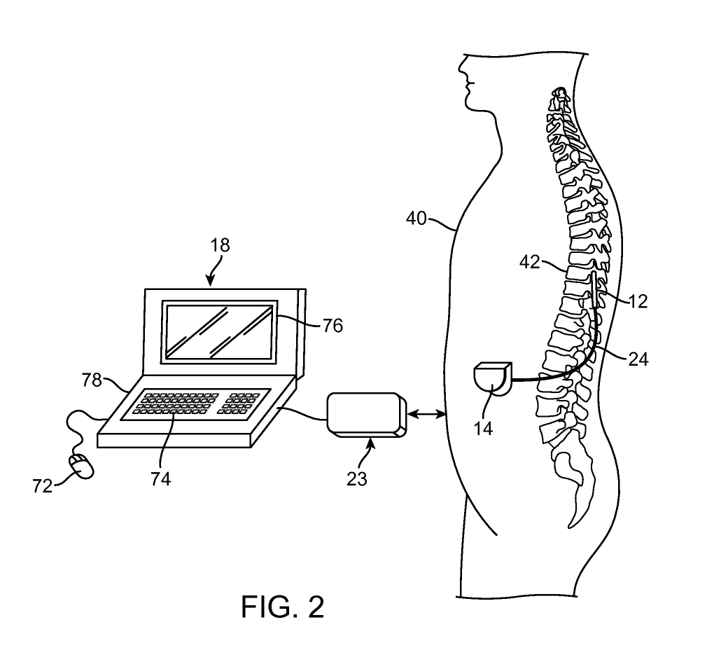

Referring to FIG. 2, the neurostimulation leads 12 are implanted within the spinal column 46 of a patient 48. The preferred placement of the neurostimulation leads 12 is adjacent, i.e., resting near, or upon the dura, adjacent to the spinal cord area to be stimulated. Due to the lack of space near the location where the neurostimulation leads 12 exit the spinal column 46, the IPG 14 is generally implanted in a surgically-made pocket either in the abdomen or above the buttocks. The IPG 14 may, of course, also be implanted in other locations of the patient's body. The lead extensions 24 facilitate locating the IPG 14 away from the exit point of the neurostimulation leads 12. As there shown, the CP 18 communicates with the IPG 14 via the telemetry wand 23. While the neurostimulation leads 12 are illustrated as being implanted near the spinal cord area of a patient, the neurostimulation leads 12 may be implanted anywhere in the patient's body, including a peripheral region, such as a limb, or the brain. After implantation, the IPG 14 is used to provide the therapeutic stimulation under control of the patient.

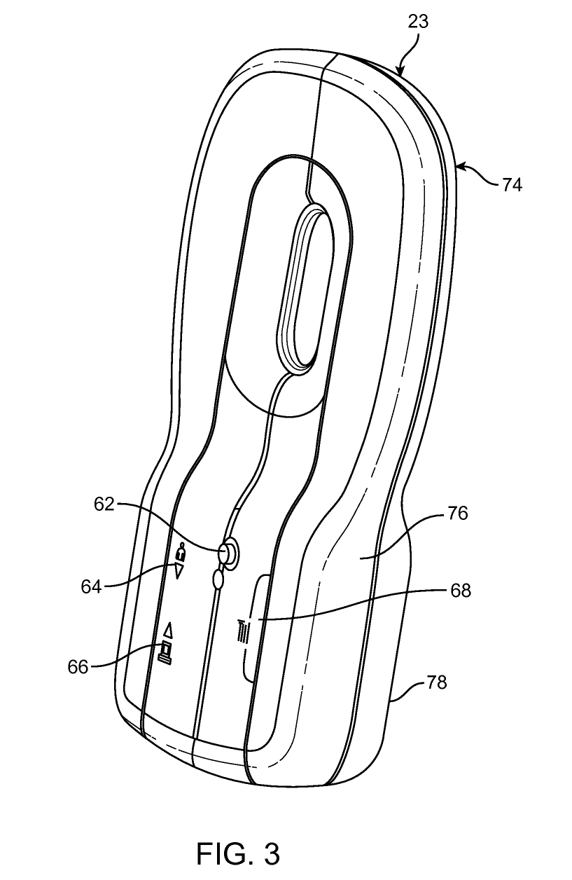

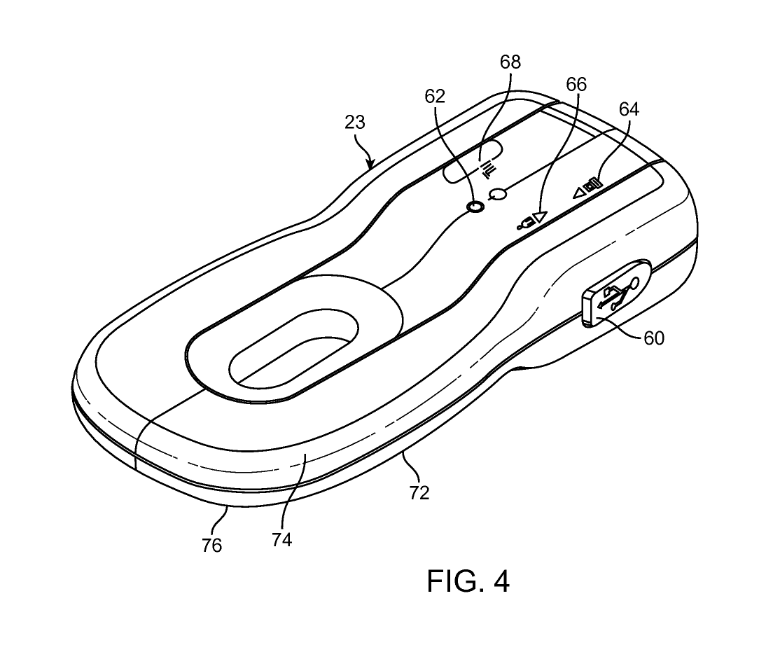

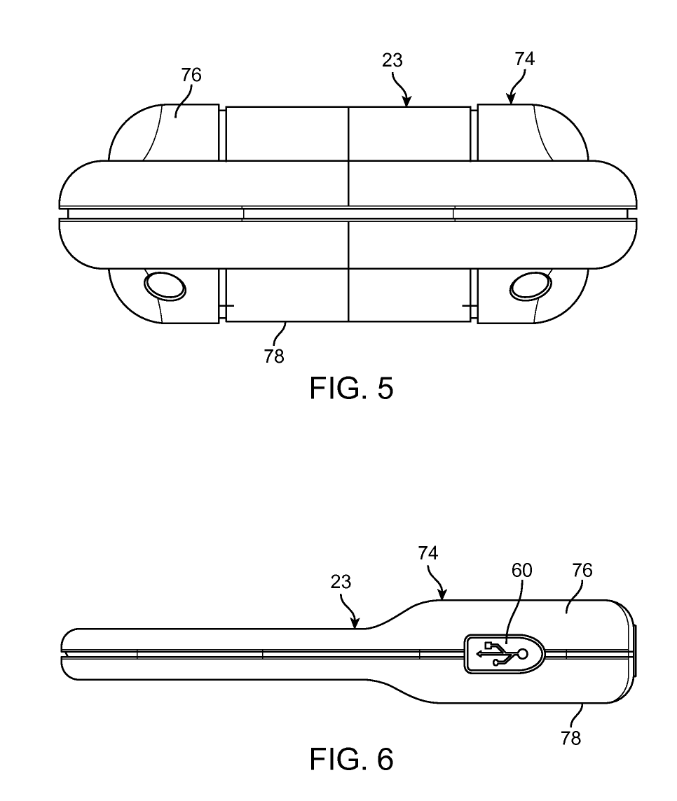

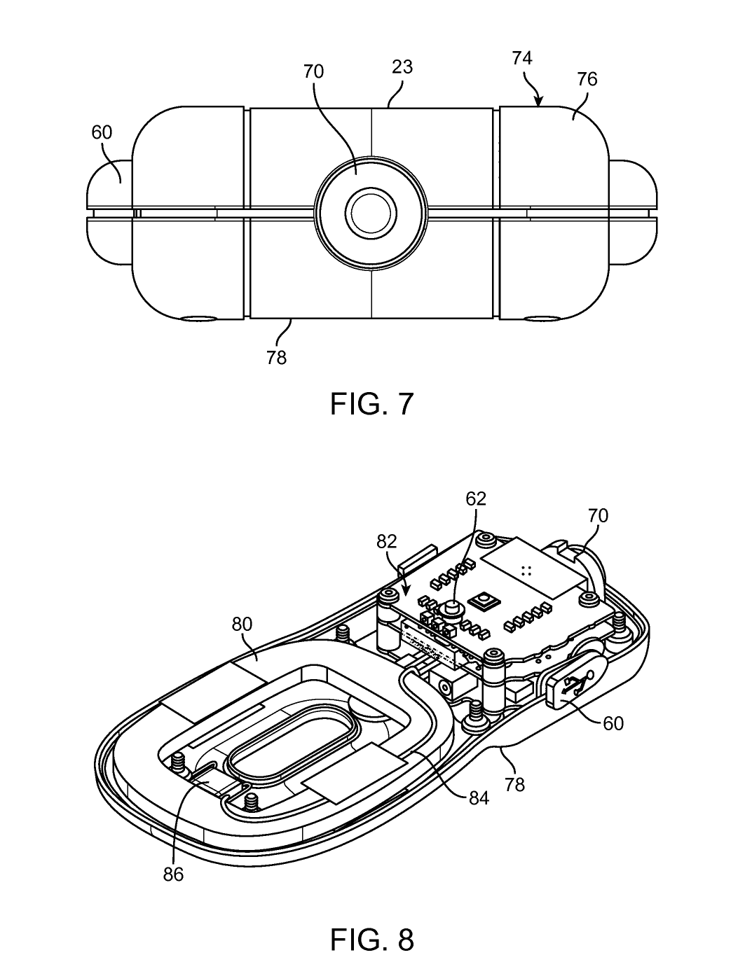

Referring to FIGS. 3-8, the telemetry wand 23 will now be described in further detail. The telemetry wand 23 is capable of being placed in proximity to the IPG 14 (e.g., within four feet), thereby allowing the CP 18 to program and receive status information from the IPG 14. The CP 18 and telemetry wand 23 have various features that facilitate a very quick setup within the operating room.

For example, the telemetry wand 23 includes a Universal Serial Bus (USB) port 60 through which programming and status data can be communicated between the CP 18 and telemetry wand 23. In this case, the telemetry cable 33 takes the form of a USB cable that includes connectors (not shown) on opposite ends to respectively connect to the USB port 60 (shown with port cover) on the telemetry wand 23 and a corresponding USB port (not shown) on the CP 18. Significantly, the CP 18 supplies power to the telemetry wand 23 via the USB port 60. Thus, since the telemetry wand 23 is powered directly through the USB port 60, it does not require batteries, thereby eliminating the need to install batteries for each programming session.

As another example, the telemetry wand 23 is a “plug-and-play” device in that it automatically connects to a host application on the CP 18 as soon as a physical connection is made between the CP 18 and the telemetry wand 23 via the telemetry cable 33. Thus, there is no need to navigate through menus in order to place the telemetry wand 23 in a “special mode” to communicate with the IPG 14.

Furthermore, the telemetry wand 23 includes various indicators for providing the communication status with the IPG 14. In particular, the telemetry wand 23 includes a power indicator 62 for indicating whether power is currently being supplied from the CP 18 to the telemetry wand 23. The telemetry wand 23 further includes an uplink indicator 64 for indicating when data is currently being transmitted from the IPG 14 to the telemetry wand 23 (and thus, to the CP 18), and a downlink indicator 66 for indicating when data is currently being transmitted from the telemetry wand 23 (and thus, from the CP 18) to the IPG 14. The telemetry wand 23 also includes a Received Signal Strength Indication (RSSI) indicator 68 for indicating the strength of the link between the telemetry wand 23 and the IPG 14. The telemetry wand 23 also includes an audio indicator (not shown) that may be used to indicate errors in the telemetry.

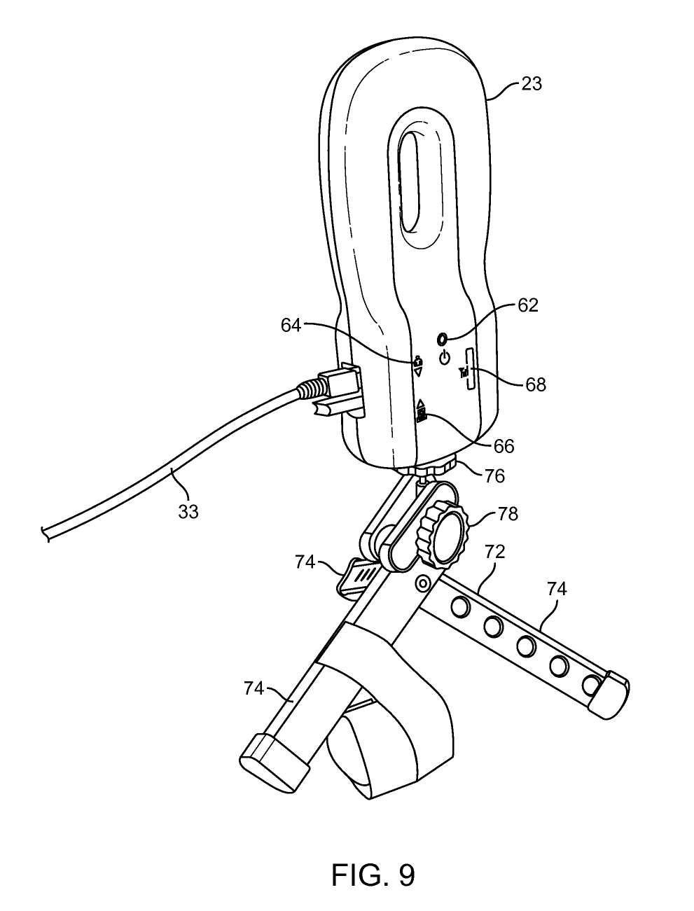

The telemetry wand 23 also includes a threaded screw receptacle 70 in which a corresponding bolt (not shown) of a mount 72 (e.g., a standard camera tripod mount) can be screwed (see FIG. 9), such that the telemetry wand 23 can be positioned upright off of the surface on which the mount 72 rests relative to the IPG 14 in any orientation. In the illustrated embodiment, the tripod mount 72 has three legs 74, a first control knob 76 for adjusting the yaw of the telemetry wand 23 (i.e., about the longitudinal axis of the telemetry wand 23 or the axis that is perpendicular to the surface on which the tripod mount 72 rests),and a second control knob 78 for adjusting the pitch of the telemetry wand 23 (i.e., about the transverse axis of the telemetry wand 23 or the axis that is parallel to the surface on which the tripod mount 72 rests). In this manner, the telemetry wand 23 may be affixed at the correct distance from, and orientation relative to, the antenna of the IPG 14, while allowing the clinician to have two free hands to operate the CP 18.

In the illustrated embodiment, the telemetry wand 23 comprises a housing 74 having a upper housing portion 76 and a lower housing portion 78 that fit together to contain the components therein. The housing 74 may be composed of a suitably rigid, electrically conductive, material, such as polycarbonate. The housing 74 is molded to have an ergonomic shape that easily fits within the palm of a user. Referring specifically to FIG. 8, the telemetry wand 23 includes an antenna 80 in the form of a coil that is configured as a radio frequency (RF) antenna, and a printed circuit board assembly 82, which is coupled to the coil 80 through an electrical conductor 84 and a capacitor 86. Although a coil is described as being used for the antenna 80, other technology may be used such as rotating field technology or an ultra high frequency (UHF) antenna.

The printed circuit board assembly 82 includes the interface electronics necessary to convey the signals between the USB connector 60 and the coil 80 and to display status at the indicators 62-66 described above.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A telemetry wand to facilitate communication between a programmer and a neurostimulator device, comprising:
an antenna;
telemetry circuitry configured for transmitting signals between the programmer and the neurostimulator device via the antenna coil;
a threaded screw receptacle configured for receiving a bolt of a conventional camera tripod; and
a housing carrying the antenna coil, the telemetry circuitry, and the threaded screw receptacle.

2. The telemetry wand of claim 1, further comprising a port configured for receiving a connector of a data cable.

3. The telemetry wand of claim 2, wherein the port is a Universal Serial Bus (USB) port.

4. The telemetry wand of claim 1, further comprising at least one indicator for indicating a communication status with the neurostimulator.

5. The telemetry wand of claim 4, wherein the at least one indicator comprises one or more of a power on/off indicator, an uplink indicator, a downlink indicator, and a Received Signal Strength Indication (RSSI) indicator.

6. The telemetry wand of claim 4, wherein the at least one indicator is a visual indicator.

7. The telemetry wand of claim 4, wherein the at least one indicator is an audio indicator.

8. The telemetry wand of claim 1, wherein the antenna comprises a coil.

9. The telemetry wand of claim 1, further comprising the conventional camera tripod.

10. A telemetry wand assembly to facilitate communication between a programmer and a neurostimulator device, comprising:
a telemetry wand; and
a mounting device configured for holding the telemetry wand off of a surface on which the mounting device rests, the mounting device having at least one control for adjusting the orientation of the telemetry wand relative to the neurostimulator device.

11. The telemetry wand assembly of claim 10, wherein the at least one control comprises a control for adjusting a yaw of telemetry wand relative to neurostimulator device.

12. The telemetry wand assembly of claim 10, wherein the at least one control comprises a control for adjusting a pitch of the telemetry wand relative to the neurostimulator device.

13. The telemetry wand assembly of claim 10, wherein the telemetry wand includes a threaded screw receptacle, and the mounting device comprises a bolt that mates with the threaded screw receptacle.

14. The telemetry wand assembly of claim 10, wherein the mount is a conventional camera tripod mount.
